(19) United States
(12) Patent Application Publication
(10) Pub. No.: US 2023/0014447 A1
(43) Pub. Date: Jan. 19, 2023

(54) Legionella Treatment Using Cannabinoids

(71) Applicant: BOTANIX PHARMACEUTICALS LIMITED, North Perth, Western Australia (AU)

(72) Inventors: Matthew Callahan, Philadelphia, PA (US); Michael Thurn, Kangaroobie (AU)

(21) Appl. No.: 17/780,662

(22) PCT Filed: Nov. 27, 2020
(86) PCT No.: PCT/AU2020/051281

§ 371 Date: May 27, 2022

(30) Foreign Application Priority Data
Nov. 29, 2019 (AU) 2019904527

(51) Int. Cl. A61K 31/05

(52) U.S. Cl. CPC A61K 31/05

(57) ABSTRACT

The use of a composition comprising a cannabinoid, that is cannabidiol, in the treatment or prevention of an infection by a Legionella bacterium such as Legionella pneumophilia.

### TECHNICAL FIELD

The present invention relates to a composition for the treatment or prevention of bacterial infections, particularly Legionella infections, comprising a cannabinoid, and a method for use thereof.

### BACKGROUND ART

Compounds with antimicrobial properties have attracted great interest in recent times as a result of an increase in the prevalence of infections caused by bacteria, resulting in serious or fatal diseases. Furthermore, the regular use of broad-spectrum antibiotic formulas has led to the increased occurrence of bacterial strains resistant to some antimicrobial compositions.

Novel antimicrobial compounds have the potential to be highly effective against these types of treatment-resistant bacteria. The pathogens, having not previously been exposed to the antimicrobial composition, may have little to no resistance to the treatment.

There is no indication that bacterial resistance to antibiotics will stop and for this reason new antibiotics and new treatment options are necessary to achieve a desirable treatment outcome in patients.

Legionella bacteria cause serious infections and are increasingly resistant to multiple drugs and, in some cases, most available antibiotics.

There is a need to provide new methods for the treatment of infections by bacteria, particularly bacteria resistant to the current antibiotic compounds available and particularly Legionella bacteria resistant to the current antibiotic compounds available. This invention seeks to provide such alternative treatment methods.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a composition comprising a cannabinoid. Preferably the composition may be used for the treatment or prevention of an infection by a Legionella bacterium. The present invention therefore provides a composition comprising a cannabinoid for the treatment or prevention of an infection by a Legionella bacterium. The invention further provides a composition comprising cannabidiol and/or acids thereof for the treatment or prevention of an infection by a Legionella bacterium.

According to another aspect of the invention, there is provided a method for the treatment or prevention of an infection by a Legionella bacterium in a subject in need of such treatment comprising the step of:

administering an effective amount of a composition comprising a cannabinoid.

The invention further provides a method for the treatment or prevention of an infection by a Legionella bacterium in a subject in need of such treatment comprising the step of:

administering an effective amount of a composition comprising cannabidiol and/or acids thereof.

The composition may be administered orally, by injection, or by nasal or pulmonary administration.

According to another aspect of the invention, there is provided the use of a composition comprising a cannabinoid for the treatment or prevention of a Legionella bacterial infection in a subject in need of such treatment or prevention. The invention further provides the use of a composition comprising cannabidiol and/or acids thereof for the treatment or prevention of a Legionella bacterial infection in a subject in need of such treatment or prevention.

According to another aspect of the invention, there is provided the use of a cannabinoid, in the manufacture of composition for the treatment of an infection by a Legionella bacterium in a subject. The invention further provides the use of cannabidiol and/or acids thereof in the manufacture of composition for the treatment of an infection by a Legionella bacterium in a subject.

According to another aspect of the invention, there is provided a kit comprising a cannabinoid for the treatment or prevention of a Legionella bacterial infection in a subject in need of such treatment or prevention. The invention further provides a kit comprising cannabidiol and/or acids thereof for the treatment or prevention of a Legionella bacterial infection in a subject in need of such treatment or prevention.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably the Legionella bacterial infection to be treated or prevented is caused by Legionella pneumophilia.

### DESCRIPTION OF INVENTION

### Detailed Description of the Invention

### Cannabinoid

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo [b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl, (+)-(3S,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl).

Cannabinoids contemplated by the present invention include:
Cannabidiol (such as 2-[(1S,6S)-3-methyl-6-prop-1-en-2-ylcyclohex-2-en-1-yl]-5-pentylbenzene-1,3-diol) and acids thereof (such as cannabidiolic acid—2,4-dihydroxy-3-[(1R,6R)-3-methyl-6-prop-1-en-2-ylcyclohex-2-en-1-yl]-6-pentylbenzoic acid);
Cannabinol (6,6,9-trimethyl-3-pentylbenzo[c]chromen-1-ol) and acids thereof (such as cannabinolic acid—1-hydroxy-6,6,9-trimethyl-3-pentylbenzo[c]chromene-2-carboxylic acid);
Δ⁹-tetrahydrocannabinol (such as (−)-Δ9-tetrahydrocannabinol) and acids thereof (such as Δ⁹-tetrahydrocannabinolic acid A—(6aR,10aR)-1-hydroxy-6,6,9-trimethyl-3-pentyl-6a,7,8,10a-tetrahydrobenzo[c]chromene-2-carboxylic acid);
11-hydroxy-Δ9-tetrahydrocannabinol (such as (±)-11-hydroxy-Δ9-tetrahydrocannabinol);
Cannabigerol (2-[(2E)-3,7-dimethylocta-2,6-dienyl]-5-pentylbenzene-1,3-diol) and acids thereof (such as cannabigerolic acid—3-[(2E)-3,7-dimethylocta-2,6-dienyl]-2,4-dihydroxy-6-pentylbenzoic acid);
Tetrahydrocannabivarin—(such as (6aR,10aR)-6,6,9-trimethyl-3-propyl-6a,7,8,10a-tetrahydrobenzo[c]chromen-1-ol) and acids thereof (such as tetrahydrocannabivarinic acid—(6aR,10aR)-1-hydroxy-6,6,9-trimethyl-3-propyl-6a,7,8,10a-tetrahydrobenzo[c]chromene-2-carboxylic acid);
Cannabichromene (2-methyl-2-(4-methylpent-3-enyl)-7-pentylchromen-5-ol);
Nantradol hydrochloride ((−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate);
Nabilone (3-(1,1-dimethylheptyl)-6,6a,7,8,10,10a-hexahydro-1-hydroxy-6,6-dimethyl-9H-dibenzo[b,d]pyran-9-one);
6,6,9-trimethyl-3-pentyl-6H-dibenzo[b,d]pyran-1-ol (such as (−)-Δ8-tetrahydrocannabinol);
(3S,4S)-7-hydroxy-Δ6-tetrahydrocannabinol-1,1-dimethylheptyl (such as (−)-(3S,4S)-7-hydroxy-Δ6-tetrahydrocannabinol-1,1-dimethylheptyl and (+)-(3S,4S)-7-hydroxy-Δ6-tetrahydrocannabinol-1,1-dimethylheptyl);
Δ8-tetrahydrocannabinol-11-oic acid ((6aR,10aR)-1-hydroxy-6,6-dimethyl-3-pentyl-6a,7,10,10a-tetrahydrobenzo[c]chromene-9-carboxylic acid);
(R)-(+)-[2,3-dihydro-5-methyl-3-(-4-morpholinylmethyl)-pyrrolo[1,2,3-de]-1,4-benzoxazin-6-yl]-1-naphthalenyl-methanone;
4-(1,1-dimethylheptyl)-2,3′-dihydroxy-6′alpha-(3-hydroxypropyl)-1′,2′,3′,4′,5′,6′-hexahydrobiphenyl;
(6aR,10aR)-9-(hydroxymethyl)-6,6-dimethyl-3-pentyl-6a,7,8,10a-tetrahydrobenzo[c]chromen-1-ol.

### Abbreviations

Cannbidiol—CBD
Cannabidiolic acid—CBDA
Cannabinol—CBN
Cannabinolic acid—CBNA
Δ⁹-tetrahydrocannabinol—THC
Δ⁹-tetrahydrocannabinolic acid A—THCA-A
Cannabigerol—CBG
Cannabigerolic acid—CBGA
Cannabichromene—CBC
Tetrahydrocannabivarin—THCV
Tetrahydrocannabivarinic Acid—THCVA

Preferably, the cannabinoid is chosen from the list comprising: cannabidiol, cannabinol, cannabinolic acid, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim. Acta, 52: 1102 (1969) and in Mechoulam et al., J. Am. Chem. Soc., 87:3273 (1965), which are hereby incorporated by reference.

### Composition

According to one aspect of the invention, there is provided a composition comprising a cannabinoid. Preferably the composition may be used for the treatment or prevention of an infection by a Legionella bacterium. There is therefore provided a composition comprising a cannabinoid for use in the treatment or prevention of an infection by a Legionella bacterium. The invention further provides a composition comprising cannabidiol and/or acids thereof for the treatment or prevention of an infection by a Legionella bacterium.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The invention therefore provides a composition comprising cannabidiol and/or acids thereof for the treatment or prevention of an infection by a Legionella bacterium.

Preferably the Legionella bacterial infection to be treated or prevented is caused by Legionella pneumophilia.

The compositions may contain more than one cannabinoid. For example, the composition of the present invention may contain a combination of two, three or more cannabinoids. In preferred forms, at least one such cannabinoid is cannabidiol.

The term “infection” as used herein means colonization by a micro-organism and/or multiplication of a micro-organism, in particular, a bacterium and more particularly a Legionella bacterium. The infection may be unapparent or result in local cellular injury. The infection may be localized, subclinical and temporary or alternatively may spread by extension to become an acute or chronic clinical infection. The infection may also be a latent infection, in which the microorganism is present in a subject, however the subject does not exhibit symptoms of disease associated with the organism.

Preferably the composition of the present invention delivers a therapeutically effective amount of the cannabinoid to the subject.

The phrase “therapeutically effective amount” as used herein refers to an amount of the cannabinoid sufficient to inhibit bacterial growth associated with bacterial carriage or a bacterial infection. That is, reference to the administration of the therapeutically effective amount of a cannabinoid according to the methods or compositions of the invention refers to a therapeutic effect in which substantial bacteriocidal or bacteriostatic activity causes a substantial inhibition of the bacterial carriage or bacterial infection. The term “therapeutically effective amount” as used herein, refers to a nontoxic but sufficient amount of the composition to provide the desired biological, therapeutic, and/or prophylactic result. The desired results include elimination of bacterial carriage or reduction and/or alleviation of the signs, symptoms, or causes of a disease, or any other desired alteration of a biological system. An effective amount in any individual case may be determined by one of ordinary skill in the art using routine experimentation. In relation to a pharmaceutical composition, effective amounts can be dosages that are recommended in the modulation of a diseased state or signs or symptoms thereof. Effective amounts differ depending on the pharmaceutical composition used and the route of administration employed. Effective amounts are routinely optimized taking into consideration various factors of a particular patient, such as age, weight, gender, etc and the area affected by disease or disease-causing microorganisms.

As used herein, “treating” or “treatment” refers to inhibiting the disease or condition, i.e., arresting or reducing its development or at least one clinical or subclinical symptom thereof, for example reducing or eliminating a bacterial infection. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician. In the context of treating a bacterial infection, the term treatment includes reducing or eliminating colonization by bacteria and/or multiplication of bacteria, preferably Legionella bacteria.

In one form of the invention, reducing or eliminating colonization by bacteria means reducing or eliminating colonization by bacteria as measured by % bacteria killed.

In one form of the invention, reducing or eliminating colonization by bacteria means reducing or eliminating colonization by bacteria as measured by a log₁₀ reduction in bacteria.

Preferably the compositions of the present invention do not contain any additional compound that removes or substantially removes or reduces the integrity of the outer membrane of a Gram-negative bacteria.

### Method of Treatment

According to another aspect of the invention, there is provided a method for the treatment or prevention of an infection by a Legionella bacterium in a subject in need of such treatment comprising the step of:

administering an effective amount of a composition comprising a cannabinoid.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The invention therefore provides a method for the treatment or prevention of an infection by a Legionella bacterium in a subject in need of such treatment comprising the step of:

administering an effective amount of a composition comprising cannabidiol and/or acids thereof.

Preferably the Legionella bacterial infection to be treated or prevented is caused by Legionella pneumophilia.

### Infections Treated by Oral Administration

In one aspect, the composition used in the method for the treatment or prevention of an infection by a Legionella bacterium in a subject in need of such treatment is an oral pharmaceutical composition comprising a cannabinoid for the treatment of an infection. Any infection in a subject by a Legionella bacterium may be treated using an orally administered treatment method.

The oral treatment method may comprise the administration of a cannabinoid to the gastrointestinal (GI) tract of the subject. Preferably, the cannabinoid enters the blood stream via absorption in the GI tract and is systemically available to the subject. However, the oral treatment method may comprise administering the cannabinoid to the GI tract for a localised effect.

### Infections Treated by Injection

In one aspect, the composition used in the method for the treatment or prevention of an infection by a Legionella bacterium in a subject in need of such treatment is an injected pharmaceutical composition for the treatment of an infection. Any infection in a subject by a Legionella bacterium may be treated using a method of treatment of injected cannabinoids.

The injection treatment method may be by intravenous injection, intramuscular injection, or intraperitoneal injection. The administration may be intraventricularly, intracranially, intracapsularly, intraspinally, or intracisternally. Preferably, the injection treatment method is by intravenous injection.

The method of injected cannabinoids may comprise the administration of a cannabinoid to the subject. Preferably, the cannabinoid enters the blood stream via IV administration or a subcutaneous bolus and is systemically available to the subject.

### Infections Treated by Nasal or Pulmonary Administration

In one aspect, the composition used in the method for the treatment or prevention of an infection by a Legionella bacterium in a subject in need of such treatment is a nasal or pulmonary pharmaceutical composition for the treatment of an infection. Any infection in a subject by a Legionella bacterium may be treated using a nasal or pulmonary delivered treatment method.

Preferably, infections of the nasal cavity, sinuses, respiratory tract and lungs are treated using a nasal or pulmonary treatment method. The nasal or pulmonary treatment method may comprise the administration of a cannabinoid to the nasal or pulmonary system of the subject. The cannabinoid may enter the blood stream via absorption in the nasal or pulmonary system and be systemically available to the subject. However, the cannabinoid dosing method may alternatively comprise administering the cannabinoid to the nasal or pulmonary system for a localised effect.

### Additional Antimicrobials

Other active agents may also be incorporated into the composition of the present invention. For example, additional antimicrobial agents such as antibacterials, antifungals etc may be incorporated. In a preferred form of the invention, the additional antimicrobial agent is not a compound that removes or substantially removes or reduces the integrity of the outer membrane of a Gram-negative bacteria.

For example, the composition may further comprise benzoyl peroxide, erythromycin, clindamycin, doxycycline or meclocycline.

Additional antimicrobial agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

### Subject

The subject may be any subject capable of infection by a bacterium, particularly a Legionella bacteria. The subject may be mammalian or avian. Preferably, the subject is selected from the group comprising human, canine, avian, porcine, bovine, ovine, equine, and feline. More preferably, the subject is selected from the group comprising human, bovine, porcine, equine, feline and canine; most preferably human.

### Use

According to another aspect of the invention, there is provided the use of a composition comprising a cannabinoid for the treatment or prevention of a Legionella bacterial infection in a subject in need of such treatment or prevention.

According to another aspect of the invention, there is provided the use of a cannabinoid, in the manufacture of composition for the treatment of an infection by a Legionella bacterium in a subject.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The invention therefore provides the use of a composition comprising cannabidiol and/or acids thereof for the treatment or prevention of a Legionella bacterial infection in a subject in need of such treatment or prevention. The invention further provides the use of cannabidiol and/or acids thereof in the manufacture of composition for the treatment of an infection by a Legionella bacterium in a subject.

Preferably the Legionella bacterial infection to be treated or prevented is caused by Legionella pneumophilia.

### Delivery

In one embodiment of the invention, the cannabinoid is administered to the subject using a dosing regimen selected from the group consisting of: three times daily; two times daily; daily; every second day, every third day, once weekly; once fortnightly and once monthly.

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject between one and five times per day, more preferably once or twice per day.

The compositions used in the treatment methods of the invention may be administered by injection, or prepared for oral, inhaled (pulmonary), nasal, or any other form of administration. Preferably the compositions are administered, for example, intravenously, subcutaneously, intramuscularly, intraventricularly, intracranially, intracapsularly, intraspinally, intracisternally, intraperitoneally, intranasally or by aerosol administration.

The mode of administration is preferably suitable for the form in which the composition has been prepared. The mode of administration for the most effective response may be determined empirically and the means of administration described below are given as examples, and do not limit the method of delivery of the composition of the present invention in any way. All the above compositions are commonly used in the pharmaceutical industry and are commonly known to suitably qualified practitioners.

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the cannabinoid to the subject. The carrier may be liquid or solid, and is selected with the planned manner of administration in mind.

The composition of the invention may be selected from the group consisting of: an immediate release composition, a delayed release composition, a controlled release composition and a rapid release composition.

The composition of the invention may further comprise an anti-inflammatory agent (such as a corticosteroid). If the composition is a topical composition, an anticomedolyic agent (such as tretinoin), and/or a retinoid or derivative thereof may also be added.

The compositions described herein may be formulated by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent (e.g. a cannabinoid) dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

The administration of the cannabinoid in accordance with the methods and compositions of the invention may be by any suitable means that results in an amount sufficient to treat a microbial infection or to reduce microbial growth at the location of infection.

The cannabinoid may be contained in any appropriate amount and in any suitable carrier substance, and is generally present in an amount of 1-95% by weight of the total weight of the composition.

The pharmaceutical composition may be formulated according to the conventional pharmaceutical or veterinary practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pa., USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the cannabinoid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

### Oral Compositions

Compositions of the invention may be administered orally.

Contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the composition will include therapeutic agents (e.g. a cannabinoid), and inert ingredients which allow for protection against the stomach environment, and release of the cannabinoid in the intestine.

For the cannabinoid of the invention, the location of release may be the stomach, the small intestine (the duodenum, the jejunum, or the ileum), or the large intestine. One skilled in the art has available compositions that will not dissolve in the stomach, yet will release the material in the duodenum or elsewhere in the intestine. In one aspect, the release will avoid the deleterious effects of the stomach environment, either by protection of the composition or by release of the cannabinoid beyond the stomach environment, such as in the intestine.

It is believed that the oral bioavailability of cannabinoids is only 4% to 12% and absorption is highly variable. Although most cannabinoids are generally easily absorbed due to their high partition coefficient (P), they are subject to degradation in the stomach and significant first-pass metabolism.

Preferably, the cannabinoid is released in the lower gastrointestinal tract.

The oral dosage method may be provided using an oral sustained release pharmaceutical composition comprising a therapeutically effective pharmaceutical composition according to the invention, and a release retardant.

In one aspect of the present invention the release retardant is a water-soluble, water swellable and/or water insoluble polymer. In particular, water-soluble polymers are selected from the group comprising are ethylcellulose, hydroxypropyl cellulose, hydroxypropyl methyl cellulose, an enteric coating; and a semipermeable membrane. In another aspect of the invention the release retardant is a non-polymeric release retardant. More particularly, the non-polymeric release retardant is hydrogenated castor oil. The compositions of the invention may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules consist of a hard shell (such as gelatin) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical composition has passed from the stomach and through until and after the pharmaceutical composition reaches the intestine. The phrase “sustained release” also means delayed release wherein release of the therapeutic compound is not immediately initiated upon the pharmaceutical composition reaching the stomach but rather is delayed for a period of time, for example, until when the pharmaceutical composition reaches the intestine. Upon reaching the intestine, the increase in pH may then trigger release of the therapeutic agent from the pharmaceutical composition.

Though term “release retardant” is used herein, means a substance that reduces the rate of release of a therapeutic agent from a pharmaceutical composition when orally ingested. The release retardant may be a polymer or a non-polymer. The release retardant may be used according to any one of several sustained release systems including, for example, a diffusion system, a dissolution system and/or an osmotic system.

In certain aspects, the therapeutic agent (e.g. a cannabinoid) is included in the composition as fine multi-particulates in the form of granules or pellets of particle size about 1 mm. The composition of the material for capsule administration is, in certain aspects, a powder, lightly compressed plug, or even as a tablet. In one aspect, the composition comprising the therapeutic agent could be prepared by compression.

Colourants and flavouring agents may optionally be included. For example, compositions may be formulated (such as, and without limitation, by liposome or microsphere encapsulation) and then further contained within an edible product, such as a refrigerated beverage containing colorants and flavouring agents.

The volume of the composition may be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the solid dosage form compositions of the present invention. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the cannabinoid together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the compositions of the invention to prevent sticking during the composition process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during composition and to aid rearrangement during compression might be optionally added. The glidants may include without limitation starch, talc, pyrogenic silica and hydrated silicoaluminate.

To aid dissolution of the therapeutic agent (e.g. a cannabinoid) into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the composition of the therapeutic agent either alone or as a mixture in different ratios.

Additives which that potentially enhance uptake of the therapeutic agent include, without limitation, the fatty acids oleic acid, linoleic acid and linolenic acid.

Controlled release composition may be desirable. In certain aspects, the therapeutic agents could be incorporated into an inert matrix that permits release by either diffusion or leaching mechanisms i.e., gums. In some aspects, slowly degenerating matrices may also be incorporated into the composition. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example, in a pan coater or in a fluidized bed or by compression coating.

### Injectable Compositions

Compositions of the invention may be administered via injection. The route of injection may be subcutaneous, intramuscular, intravenous, intraperitoneal, intracardiac, intraarticular, or intracavernous injection.

The compositions suitable for injectable use optionally include sterile aqueous solutions (where water-soluble) or dispersions and sterile powders for the extemporaneous preparation of sterile injectable solutions or dispersion. Alternatively, the therapeutic agents of the invention are, in certain aspects encapsulated in liposomes and delivered in injectable solutions to assist their transport across cell membrane. Alternatively or in addition such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminium monostearate and gelatine.

The invention also provides an injectable sustained release pharmaceutical composition comprising a therapeutically effective pharmaceutical composition according to the invention, and a release retardant. The release retardant may be, for example, aluminium monostearate and gelatine.

Sterile injectable solutions are prepared by incorporating the therapeutic agents in the required amount in an appropriate solvent with various other ingredients enumerated above, as required, followed by filtered sterilisation. Generally, dispersions are prepared by incorporating the various sterilised therapeutic agents into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the therapeutic agents plus any additional desired ingredient from previously sterile-filtered solution thereof.

### Nasal and Pulmonary Compositions

Compositions of the invention may be administered via nasal or pulmonary delivery.

A wide range of mechanical devices designed for pulmonary delivery of therapeutic agents exist, including but not limited to nebulizers, metered-dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of compositions suitable for the dispensing of the cannabinoid. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Compositions suitable for use with a nebulizer, either jet or ultrasonic, will typically comprise the cannabinoid suspended in water or a non-aqueous solvent. The composition may also include a buffer and a simple sugar (e.g., for stabilization and regulation of osmotic pressure). The nebulizer composition may also contain a surfactant, to reduce or prevent surface induced aggregation of the cannabinoid caused by atomization of the solution in forming the aerosol.

Compositions for use with a metered dose inhaler device will generally comprise a finely divided powder containing the cannabinoid suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the cannabinoid and may also include a bulking agent, such as lactose, sorbitol, sucrose, or mannitol in amounts which facilitate dispersal of the powder from the device, e.g., 50 to 90% by weight of the composition. The cannabinoid should most advantageously be prepared in particulate form with an average particle size of less than 10 microns, most preferably 0.5 to 5 microns, for most effective delivery to the distal lung.

Nasal delivery of a cannabinoid in the treatment methods of the present invention is also contemplated. Nasal delivery allows the passage of the cannabinoid to the blood stream directly after administering the therapeutic product to the nose, without the necessity for deposition of the cannabinoid in the lung. Compositions for nasal delivery include those with dextran or cyclodextran.

### Kits

The invention also provides kits for use in the instant methods. There is therefore provided a kit comprising a cannabinoid for the treatment or prevention of a Legionella bacterial infection in a subject in need of such treatment or prevention. The invention further provides a kit comprising cannabidiol and/or acids thereof for the treatment or prevention of a Legionella bacterial infection in a subject in need of such treatment or prevention.

Kits of the invention include one or more containers comprising a cannabinoid as described herein, and instructions for use in accordance with any one of the methods described herein. The kit may further comprise a description of selecting an individual suitable for treatment based on identifying whether that individual has an infection by a Legionella bacterium. The kit may further comprise a description of administering a cannabinoid as described herein to an individual at risk of developing an infection by a Legionella bacterium.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably the Legionella bacterial infection to be treated or prevented is caused by Legionella pneumophilia.

The instructions generally include information as to dosage, dosing schedule, and route of administration for the intended treatment. The containers may be unit doses, bulk packages (e.g. multi-dose packages) or sub-unit doses. Instructions supplied in the kits of the invention are typically written instructions on a label or package insert. The label or package insert indicates that the composition is used for treating, ameliorating and/or preventing an infection by a Legionella bacterium. Instructions may be provided for practising any of the methods described herein.

### General

Those skilled in the art will appreciate that the invention described herein is susceptible to variations and modifications other than those specifically described. The invention includes all such variation and modifications. The invention also includes all of the steps, features, compositions and therapeutic agents referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, compositions and methods are clearly within the scope of the invention as described herein.

The invention described herein may include one or more range of values (e.g. size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” or “therapeutic agent” may mean one active agent or therapeutic agent, or may encompass two or more active agents or therapeutic agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

In this study, to evaluate the spectrum of activity, cannabidiol (CBD) was profiled against Legionella. Testing was conducted following guidelines recommended by the Clinical and Laboratory Standards Institute (CLSI, 1-4), using vancomycin, meropenem, levofloxacin, and gentamicin as comparators for testing aerobic bacteria.

### Materials and Methods

### Test Agents

Test agents were supplied by Micromyx. Stock solutions of all compounds were prepared on the first day of testing at 101× the highest test concentration using solvents recommended by CLSI. CBD was prepared fresh on each of testing. Information regarding compound source, lot number, testing concentrations and drug diluent for the comparators and test agents is detailed below:

| | | | Testing Conc | |
|---|---|---|---|---|
| Test agent | Supplier | Lot No. | Range (μg/mL) | Solvent/Diluent |
| CBD | Cerilliant | FE12271801 | 64-0.06 | DMSO/DMSO |
| Vancomycin | Sigma | 080M1341V | 32-0.03 | Water/Water |
| Clindamycin | Sigma | 021M1533 | 32-0.03 | Water/Water |
| Metronidazole | Sigma | 095K0693 | 32-0.03 | DMSO/Water |
| Gentamicin | Sigma | SLBT5354 | 32-0.03 | Water/Water |
| Meropenem | USP | J0K434 | 32-0.03 | Water/Water |
| Levofloxacin | Sigma | BCBF7004V | 32-0.001 | 1/2 volume of water, |
| | | | | then0.1M NaOH dropwise to |
| | | | | dissolve/Water |

### Organisms

The test organisms evaluated in this study consisted of clinical isolates from the Micromyx Repository, reference isolates from the American Type Culture Collection (ATCC; Manassas, Va.), isolates from the antibiotic resistance bank from the Centers for Disease Control and Prevention (CDC; Atlanta, Ga.), and isolates from the Network on Antimicrobial Resistance in Staphylococcus aureus (NARSA; BEI Resources, Manassas, Va.). Upon initial receipt at Micromyx, the organisms were sub-cultured onto an appropriate agar medium. Following incubation, colonies were harvested from these plates and cell suspensions prepared and frozen at −80° C. with a cryoprotectant.

Prior to testing, Legionella was grown on CYE Agar (Oxoid, Hampshire, UK, Lot 2280778) supplemented with Legionella Growth Supplement BCYE (HiMedia, Mumbai, India; Lot 0000337458). Aerobic bacteria were incubated at 35° C. overnight.

### Test Media

Buffered Yeast Extract Broth (BYEB) was prepared to test the MIC of the Legionella isolates. For 1 L of this medium, 10 g H₂NCOCH₂NHCH₂CH₂SO₃H (ACES; Alfa Aesar, Haverhill, Mass.; Lot 10185658), 10 g yeast extract (Oxoid; Lot 1424987-02), 1 g alpha ketoglutarate (Sigma; Lot BCBX4691), 0.4 g L-cysteine (Sigma; Lot. No. BCBQ4116V) and 0.25 g Iron (Ill) pyrophosphate (Sigma; Lot No. SLBK9182V) were dissolved in H₂O, pH-adjusted to 6.9 using 1 N NaOH, and filter-sterilized using a 0.22 μm filter.

### Broth Microdilution MIC Testing

MIC values were determined using a broth microdilution procedure described by CLSI (1-4). Automated liquid handlers (Multidrop 384, Labsystems, Helsinki, Finland; Biomek 2000 and Biomek FX, Beckman Coulter, Fullerton, Calif.) were used to conduct serial dilutions and liquid transfers.

To prepare the drug mother plates, which would provide the serial drug dilutions for the replicate daughter plates, the wells of columns 2 through 12 of standard 96-well microdilution plates (Costar 3795) were filled with 150 μl of the designated diluent for each row of drug. The test article and comparator compounds (300 μl at 101× the highest concentration to be tested) were dispensed into the appropriate wells in column 1. The Biomek 2000 was then used to make 2-fold serial dilutions in the mother plates from column 1 through column 11. The wells of column 12 contained no drug and served as the organism growth control wells for the assay.

The daughter plates were loaded with 190 μL per well of the appropriate test medium for the tested organism using the Multidrop 384. The daughter plates were completed on the Biomek FX instrument which transferred 2 μL of drug solution from each well of a mother plate to the corresponding well of each daughter plate in a single step.

A standardized inoculum of each test organism was prepared per CLSI methods (1-4). The inoculum for each organism was dispensed into sterile reservoirs divided by length (Beckman Coulter), and the Biomek 2000 was used to inoculate the plates. Daughter plates were placed on the Biomek 2000 work surface in reverse orientation so that inoculation took place from low to high drug concentration. Plates were inoculated with 10 μL of the inoculum resulting in a final cell density of approximately 5×10⁵ CFU/mL per well.

The plates for testing of aerobic organisms were stacked 3 to 4 high, covered with a sterile lid, and incubated at 35° C. under the appropriate conditions for the time periods specified in CLSI (1-4). Plates were incubated for both 24 and 48 hr for Legionella.

Following incubation, the microplates were removed from the incubator and viewed from the bottom using a plate viewer. For each of the test media and each drug, an un-inoculated solubility control plate was observed for evidence of drug precipitation and media sterility. The MIC was read and recorded as the lowest concentration of drug that inhibited visible growth of the organism.

### Results

CBD was profiled against Legionella. The in vitro activity of CBD and the comparators vancomycin, levofloxacin, meropenem and gentamicin against Gram-positive aerobic bacteria is shown is Table 1. Quality control testing of comparator agents against QC organisms were within CLSI published QC ranges. CBD displayed MIC values of 1 μg/mL against isolates of Legionella pneumophila.

TABLE 1
In vitro activity of CBD and comparators against Gram-negative
aerobic bacteria

| | MMX or | | | | | |
|---|---|---|---|---|---|---|
| Bacterial Isolates | ATCC No. | VAN | LVX | MEM | GEN | CBD |
| Legionella | MMX 7515 | 0.12* | 0.5* | 0.25* | 1* | 1* |
| pneumophila | | | | | | |
| L. pneumophila | MMX 7515 | 0.25** | 0.5** | 0.5** | 2** | 1 ** |

VAN, vancomycin;
LVX, levofloxacin;
MEM, meropenem;
GEN, gentamicin
*plate read at 24 hr
**plate read at 48 hr

### REFERENCES

1. Clinical and Laboratory Standards Institute (CLSI). Methods for Dilution Antimicrobial Susceptibility Tests for Bacteria That Grow Aerobically; Approved Standard—Eleventh Edition. CLSI document M07-Δ11. CLSI, 950 West Valley Road, Suite 2500, Wayne, Pa. 19087 USA, 2018.
2. CLSI. Performance Standards for Antimicrobial Susceptibility Testing; 29th ed. CLSI supplement M100. CLSI, 950 West Valley Road, Suite 2500, Wayne, Pa. 19087 USA, 2019.
3. CLSI. Methods for Antimicrobial Susceptibility Testing of Anaerobic Bacteria; Approved Standard—Ninth Edition. CLSI document M11-Δ9. CLSI, 950 West Valley Road, Suite 2500, Wayne, Pa. 19087 USA, 2018.
4. CLSI. Methods for Antimicrobial Dilution and Disk Susceptibility Testing of Infrequently Isolated or Fastidious Bacteria; 3rd ed. CLSI guideline M45. CLSI, 940 West Valley Road, Suite 2500, Wayne, Pa. 19087 USA, 2016.

1. A composition comprising a cannabinoid for use in the treatment or prevention of an infection by a Legionella bacterium.

2-9. (canceled)
